(19) United States
(12) Patent Application Publication
(10) Pub. No.: US 2016/0162654 A1
(43) Pub. Date: Jun. 9, 2016

(54) SYSTEMS AND METHODS FOR OVULATION STATUS AND PREGNANCY DETECTION

(71) Applicants: Vinay Dattaprabhakar Gokhale, Laguna Niguel, CA (US); Erik Peter Raith, Laguna Niguel, CA (US); John Scot Sinclair, Laguna Niguel, CA (US)

(72) Inventors: Vinay Dattaprabhakar Gokhale, Laguna Niguel, CA (US); Erik Peter Raith, Laguna Niguel, CA (US); John Scot Sinclair, Laguna Niguel, CA (US)

(21) Appl. No.: 14/560,946

(22) Filed: Dec. 4, 2014

(51) Int. Cl. G06F 19/00; H04L 29/08; H04L 29/06; G01N 33/76; H04W 76/02

(52) U.S. Cl. CPC G06F 19/3418; G01N 33/76; H04W 76/02; H04L 65/403; H04L 67/125; G06F 19/366

(57) ABSTRACT

Systems and methods are disclosed for detecting pregnancy or ovulation status by detecting a wireless connection between a pregnancy test sensor and a mobile device; if the wireless connection is made: displaying guidance information on the mobile device for a user to dip a strip into a liquid; detecting at the sensor the presence of liquid on the strip and color change on the strip; and displaying on the mobile device one or more multimedia presentations based on personalized options relating to pregnancy detection.

### FIELD OF THE INVENTION

The present invention relates to testing devices, and in particular to ovulation status and pregnancy test devices.

### BACKGROUND OF THE INVENTION

At home ovulation and pregnancy tests are used more often today to help women and their partners plan for a successful pregnancy in conjunction with professional medical care. As more and more information becomes available about diets and behaviors that could be harmful to a child in utero, women have become more concerned about knowing when they may be pregnant in earlier stages so that they may manage their diet and behavior to a healthier lifestyle for their child. Couples suffering from impaired fertility may choose to use an ovulation test to help them conceive. A number of ovulation and pregnancy tests have been introduced to the market to help a woman to conceive and detect whether or not she is pregnant.

U.S. Pat. No. 4,033,723 discloses a method and device for detecting pregnancy. The test involves concentration by ultrafiltration of a sample of urine or serum from a subject; followed by determining the presence of human chorionic gonadotropin or of its β-subunit in the concentrated sample.

U.S. Pat. No. 4,123,509 discloses a method and device for detecting pregnancy. The test involves concentration by ultrafiltration of a sample of urine or serum from a subject; followed by determining the presence of human chorionic gonadotropin or of its β-subunit in the concentrated sample.

U.S. App. No. 20070292969 discloses a pregnancy test device that aids a woman in determining whether or not she is pregnant. The pregnancy test device includes a molded body, having a series of apertures and a display window; a sample pad, disposed within the molded body for receiving a urine sample, said sample pad treated to detect the presence of human chorionic gonadotropin or its β-subunit, and exposed through the series of apertures in the molded body; a membrane, operatively connected to the sample pad, to display the results of the pregnancy test, and said membrane positioned to be viewed through the display window of the molded body; and gold, disposed between the sample pad and the membrane.

Published Application US20130041997 discloses the architecture of an internet of things (IOT). The architecture includes multiple levels of IOT service platforms, wherein a superordinate IOT service platform is configured to manage one or more of the following function entities: IOT terminal, IOT terminal gateway, subordinate IOT service platform, special service platform and service gateway. The present invention also discloses a method for implementing an IOT service. The method includes the steps of: the superordinate IOT service platform providing management for one or more of subordinate IOT service platform, special service platform and service gateway, wherein the management includes one or more of: registration, login, logout, data synchronization and heartbeat. With the present invention, the deployed industries or special service platforms can be integrated into a unified architecture, thus lightening the burden of the IOT service platforms.

Published Application US20140241354 relates to enabling communication among one or more Internet of Things (IoT) device groups. In particular, various heterogeneous IoT devices that may need to interact with one another in different ways may be organized into IoT device groups to support efficient interaction among the IoT devices. For example, pre-defined IoT device groups may be formed organize certain IoT devices that perform similar activities and certain IoT devices may be dynamically allocated to ad-hoc IoT device groups for certain contexts (e.g., the ad-hoc IoT device groups may include IoT devices that can implement a desired function and therefore be dynamically formed to implement the desired function). Furthermore, the IoT groups may communicate hierarchically, wherein messages may be exchanged among IoT group owners or ranking members to support efficient communication between different IoT groups.

Current ovulation and pregnancy testing devices provide for a woman to void on a wick that transfers the urine to a sample pad which reacts with the urine to determine the presence and concentration of certain hormones (for example, Human Chorionic Gonadotropin (HCG) or Luteinizing (LH) hormones). Correct operation of these devices result in a very simple display of the test results, on the test device itself. Typically these results were shown to the User as color bands on a piece of paper or on a small LCD screen on the test device.

Heretofore, ovulation and pregnancy test devices were used in a stand alone fashion so that one set of test results could not be easily compared to another set of results. Additionally, aside from the test result itself, the devices were not able to provide any additional contextual information such as instruction on how to successfully conduct the test, reason why a conducted test may be invalid, answers to common questions a woman may have about the test or educational information pertinent to ovulation and pregnancy. Further, these devices are incapable of electronically sharing or results of the test with other consumers. These devices were incapable of recording the reaction of the test results from either the User or a set of individuals known to the User. Lastly, these devices were unable to utilize computing power that could improve accuracy, reliability or clarity of the test results that was not already on the device itself.

### SUMMARY

Systems and methods are disclosed for ovulation status or pregnancy tests that incorporate a wireless connection between an ovulation or pregnancy test sensor and a series of electronic devices including a SmartPhone and cloud data servers. These tests would enable the User to: 1) easily capture test data and retrieve historical data, 2) Share the results of the test with others, 3) Provide rich contextual information including Education, Medical references, Test instruction, Test diagnosis, 4) Record User reaction or reaction of others to the test results, 5) Quickly and easily enable the purchase and delivery of consumables related to the test including but not limited to procuring more of the tests themselves, and, 6) Leverage computing power not on the test device to help make the test more accurate, more reliable or more clear. These system and methods would be controlled directly by the User to protect privacy and security of the collected data. Further each enablement would itself be controlled by the User.

Advantages of the system may include one of the following:
enable capturing user reaction (ie, Selfie) or reaction of others to the test result.
Sharing the captured reaction (whether using video, audio, text or images) with others.
Improving the accuracy, reliability and clarity of the results by utilizing computational resources that are not available on the Sensor, but available in other elements of the proposed Testing system.
Providing historical results in a manner that can aid the User in understanding the current result
Providing contextual information including but not limited to ovulation and pregnancy education, testing instructions, references for professional medical care, test diagnosis, advertisements for consumables or other non-test related items
Quickly and easily enabling the purchase and deliver of more consumables related to the test
Given the User full control to enable or disable all services, manage privacy settings and destroy all previously recorded personally identifiable information

### BRIEF DESCRIPTION OF THE DRAWINGS

FIG. 1A shows an exemplary system utilizing an ovulation/pregnancy test device connected to a mobile computing and Cloud server system.

FIG. 1B shows an exemplary process to launch a connected ovulation/pregnancy test

FIG. 1C shows an exemplary process to run a pregnancy test

FIG. 1D shows an exemplary cloud and infrastructure architecture that will support a connected ovulation and pregnancy test.

FIG. 2A shows an exemplary process to connect a pregnancy test device to a mobile computing system.

FIG. 2B shows an exemplary process to handle a failed connection for FIG. 2A.

FIG. 3 shows an exemplary process to start a pregnancy test.

FIG. 4 shows an exemplary process to handle the results of the pregnancy test.

FIGS. 5 and 6 show an exemplary process to handle a positive pregnancy result.

FIGS. 7A-7B shows an exemplary process operated by the test device.

FIG. 8 shows an exemplary test device.

### DESCRIPTION

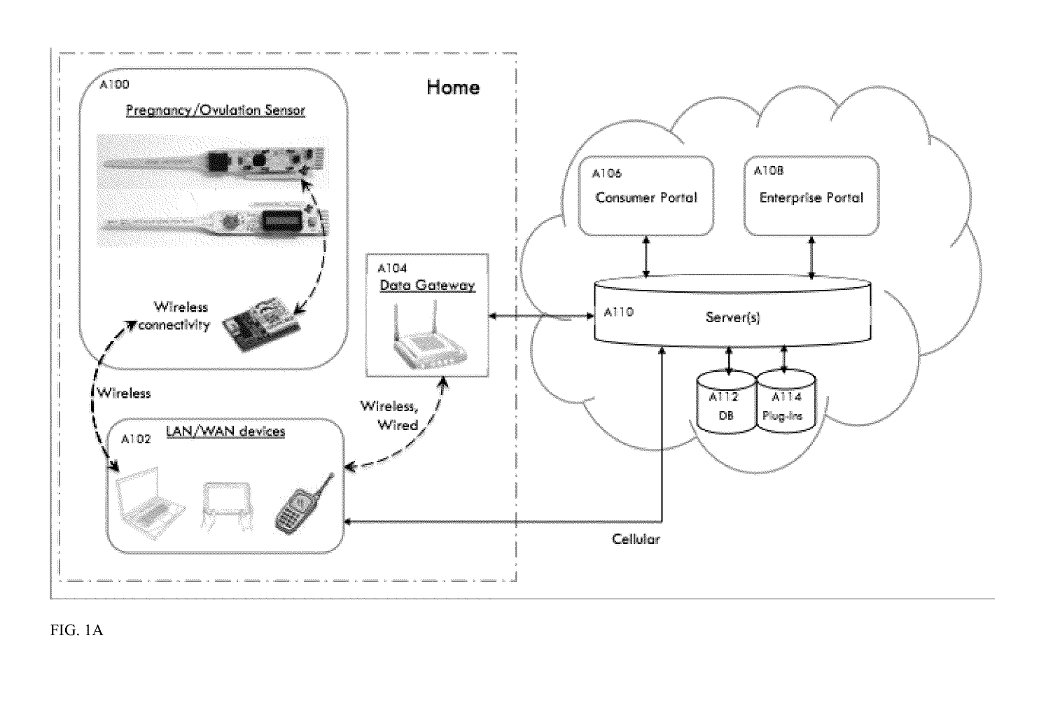

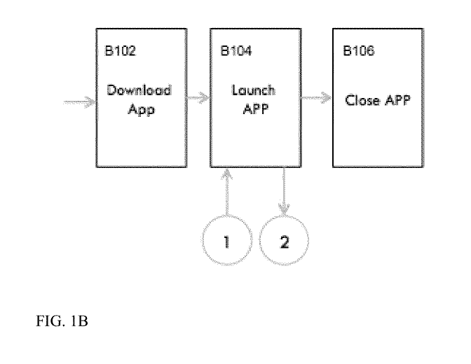

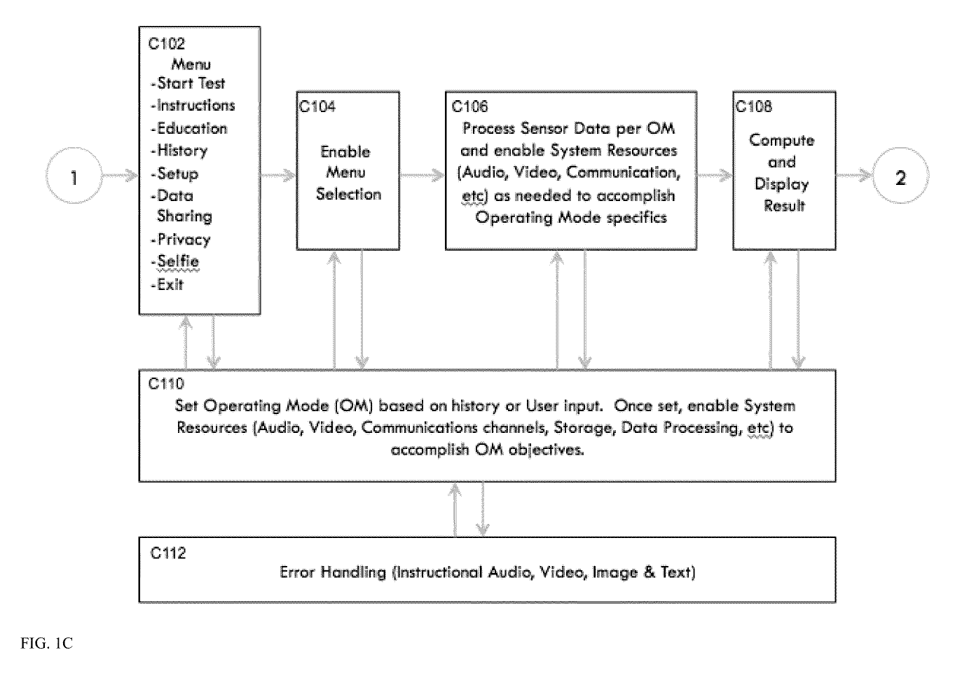

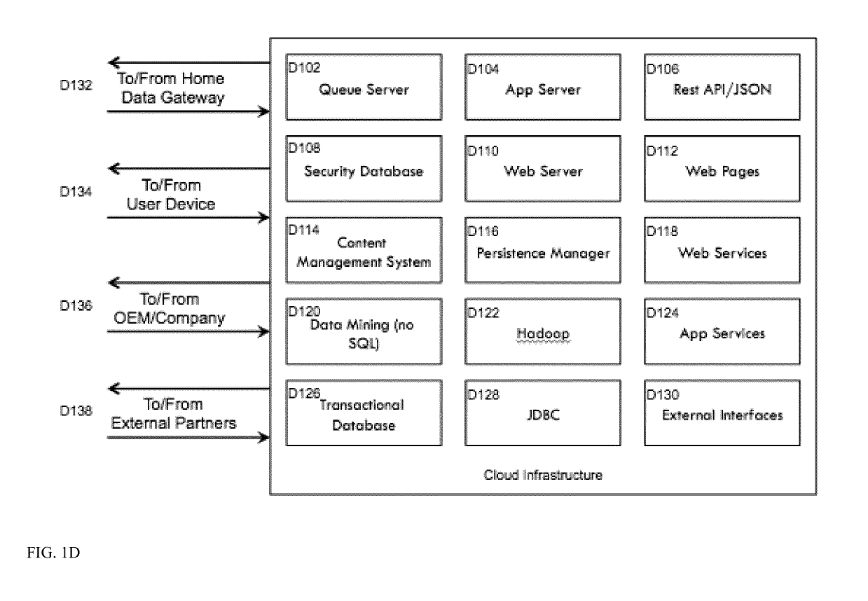

FIG. 1A shows an exemplary system utilizing an ovulation/pregnancy test device connected to a mobile computing and Cloud server system. The dotted line represents those activities that could take place in the Home. The connected pregnancy/ovulation sensor (A100) is represented as plastic casings, printed circuit board, silicon devices, Liquid Crystal Display (LCD), battery, buttons and a wireless connectivity module. The wireless protocol may be WiFi, Bluetooth, Zigbee, a derivative there of or some other substantially different protocol. The sensor (A100) communicates to a Local Area Network (LAN) or Wide Area Network (WAN) device such as a smartphone, a tablet, a laptop, a desktop (A102) over the same wireless network as enabled in the Sensor (A100). Communication between the Sensor (A100) and LAN/WAN devices (A102) is bidirectional. The LAN/WAN devices (A102) in turn communicate to either a Data Gateway (A104) over either a wired or wireless connection. The Data Gateway (104) may support wired connectivity (such as Ethernet, Universal Serial Bus, variants thereof or some other wired specification) or wireless connectivity (such as BlueTooth, WiFi, variants thereof or some other wireless specification). LAN/WAN devices (A102) may also communicate directly to cloud servers (A110) over a cellular connection. The cellular connection can by CDMA, GSM, 2G, 3G, 4G, WiMax, variants thereof or some other cellular technology. The cloud server (A110) may itself be comprised of several distinct data servers that are geographically dispersed. The cloud server (A110) in turn communicates with a Consumer Portal server (A106) or an Enterprise portal server (A108) to relay information to consumers or to Original Equipment Manufacturers (OEMs) or Enterprises. The cloud server (A110) may also be connected to one of several different types of Databases (A112) that act as repositories for data and enable storage and retrieval of stored data. Additionally, the cloud server (A114) may also be connected to several plug-in servers (A114) that enable communication with third party services such as weather, social media, web services, computing resources, content delivery networks, messaging networks, etc.

FIG. 1B shows an exemplary process to download, launch and operate a connected ovulation/pregnancy test application. The application is downloaded (B-102) from an application store such as Google Playstore, Apple App Store or such other application distribution mechanism on to a smartphone, tablet or other such mobile or wearable computing platform. The application is then launched (B-104) by the User on the smartphone or similar computing platform. After launching, the application will follow a series of options set by the User and the program as shown in FIG. 1C. After the User is finished with the application, the User will close the App (B-106). All data generated by the test or the User may or may not be stored locally within the App local storage system and/or the cloud servers (A-110), cloud content management system (D-114) or some other suitable repository as per the User direction.

FIG. 1C shows an exemplary process to run a connected pregnancy or ovulation test. After the application is launched (B-104), the User may have a menu structure (C-102) that will allow the User via a menu selection module (C-104) to select an option. The app will then process the menu selection (C-106) by utilizing whatever resources are available to the application and then display results (C-108). The results may also be stored in the cloud server (A-110). As the ovulation or pregnancy test application is run, the app will utilize a parallel process (C-110) to set operating modes, enable system resources such as audio, video, communication, storage, data processing and other similar resources to perform the tasks selected by the User. All error handling that is required to guide the User will also be done as a parallel process (C-112) and may result in instructional audio, video, image or textual information that is presented to the User.

FIG. 1D shows an exemplary cloud and infrastructure architecture that will support a connected ovulation or connected pregnancy test. No attempt is made to exhaustively list all elements within the cloud infrastructure required for such tests. Additionally, certain elements depicted in FIG. 1D can be omitted depending upon specific implementation constraints. Inputs to and Outputs from the cloud infrastructure come from four or more sources; 1) The Data Gateway, shown as FIG. 1A-104 (Fig D-132), 2) User devices, shown as Fig A-102 (Fig D-134) or User web portals, shown as Fig A-106 (Fig D-134), 3) OEM or Enterprise users, shown as FIG. 1A-108 (Fig D-136), 4) External parties, shown as FIG. 1A-114 or FIG. 1D-138 or others. The cloud infrastructure architecture is comprised of several elements and capabilities that may be interdependent as depicted in FIG. 1D.
The Queue Server (D-103) represents the ability for the cloud infrastructure to manage load balancing and overall system latency by being able to direct traffic to other entities in the system.
The App Server (D-104) is responsible for managing the authenticated delivery of all mobile Apps to user devices and managing interaction between the mobile App and the cloud infrastructure.
The Rest API/JSON (D-106) is an entity that manages Representational State Transfer and JavaScript Object Notation interfaces for the Cloud as well as other interfaces to enable intermittent network connections as well as the ability to interact with a number of different types of User devices.
The Security Database (D-108) is the logical entity for all data that may be subject to specific policy or regulatory requirements.
The Web Server (D-110) entity is responsible for managing the authenticated interaction between the User and cloud infrastructure for all web browser based interaction.
The Web Pages (D-112) entity stores all web pages that could be rendered on many different User device formats.
The Content Management System (D-114) represents the entity that interfaces with one or more 3^{rd} party content distribution systems and manages transactions that relate to large data files.
The Persistence Manager (D-116) entity enables search and query operation while simultaneously updating the databases within the system.
The Web Services (D-118) entity represents several different services such as email, network time, short messaging service, social media, enterprise specific and others that enable the cloud infrastructure to work with other established web services.
The Data Mining (D-120) entity is specialized to enable fast queries while maintaining persistence of old data as new data is recorded.
The Hadoop (D-122) entity enables the processing of large data sets within the Cloud infrastructure.
The App Services (D-124) enables a variety of services for the App to run code modules within the server cloud that can interact with other entities within the server and produce results or modify data.
The Transactional Database (D-126) enables recording of all Cloud transactions, can provide audit traces as well as record management for the System.
The JDBC (D-128) entity that enables Java DataBase Connectivity and enables database management systems within the cloud infrastructure.
The External Interface (D-130) entity represents other programming interfaces, protocols and modules that are unnamed but necessary for the cloud infrastructure to operate.
   The entities listed in FIG. 1D utilize a number of different protocols such as REST (Representation State Transfer), SOAP (Simple Object Access Protocol), HTTP (Hypertext Transfer Protocol), HTTPS (Hyptertext Transfer Protocol Secure, UDP (User Datagram Protocol) and others to enable cloud infrastructure functionality.

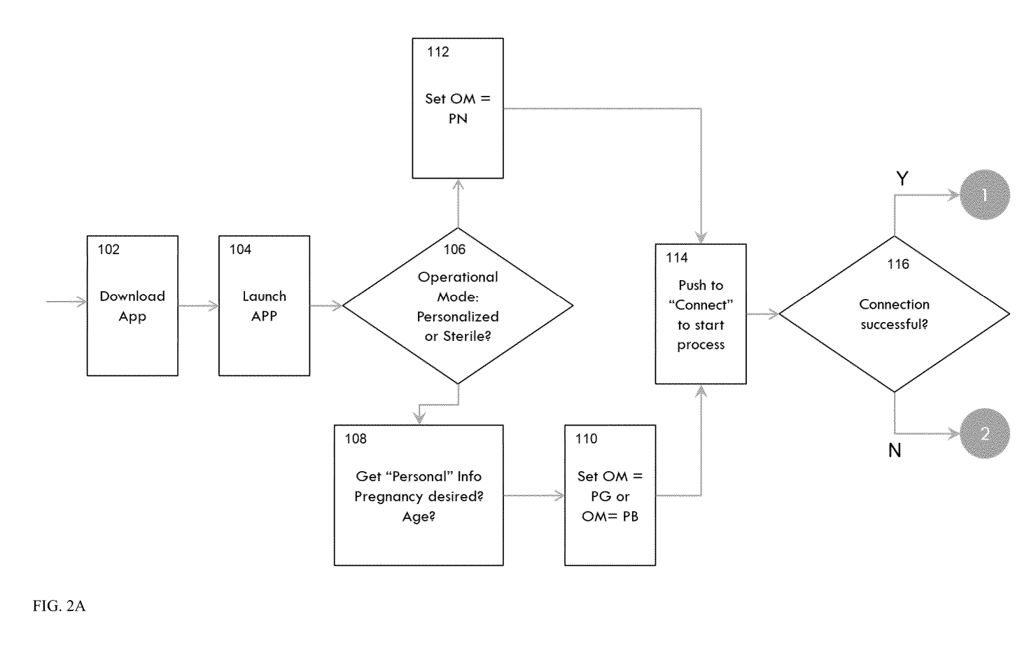

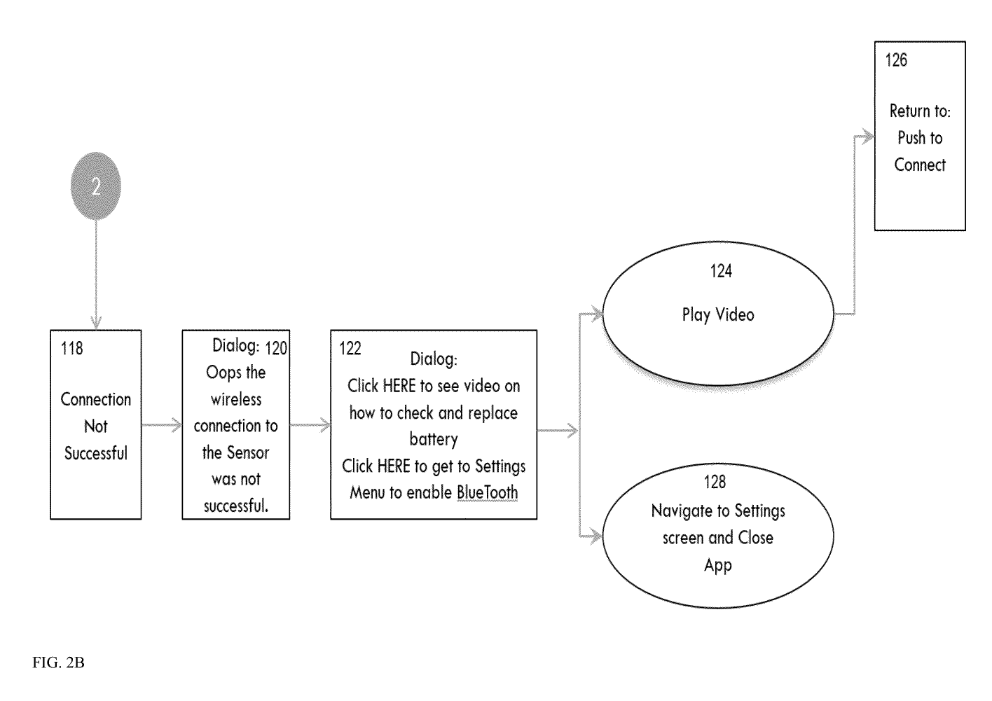

FIG. 2A shows an exemplary process to connect a pregnancy test device to a mobile computing system. First, the user downloads an application (app) to a mobile computer such as a smart phone, for example (102). Next, the user launches the app (104). Next, the user selects an operational mode of either personalized mode or sterile mode (106). In the personal mode, the process obtains personal information and age data, among others (108), and the process sets OM to PG or PB (110). Alternatively, if sterile mode is selected, the process sets OM to PN (112). The process then waits for the user to push a Connect button to start the process. If the connection is successful, the process proceeds to connector 1 and otherwise to connector 2.

FIG. 2B shows an exemplary process to handle a failed connection for FIG. 1. From connector 2, the process determines that the connection was not successful if the wireless handshake fails (118). The process presents a dialog that the wireless connection to a pregnancy sensor in the test device was unsuccessful (120). The process presents a dialog with a help video on battery check and replacement (122). The process also provides access to a setting menu to enable the wireless transceiver such as Bluetooth, for example. From there a help video can be shown (124) and the process can prompt the user to push to connect (126). Alternatively, the user can navigate to the settings screen to close the app (128).

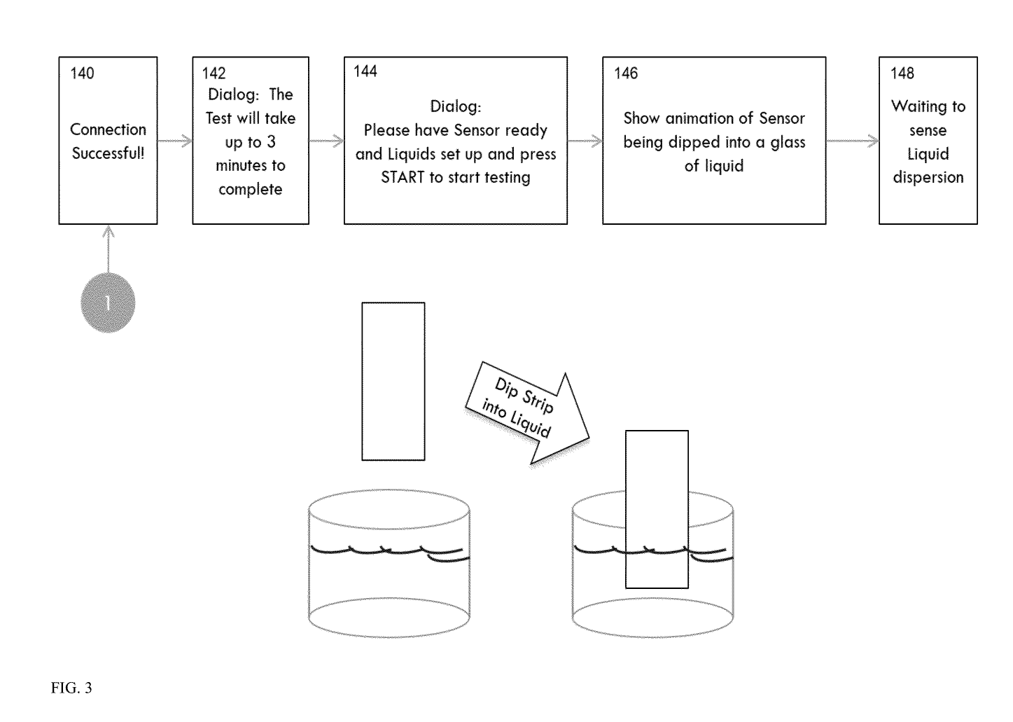

FIG. 3 shows an exemplary process to start a pregnancy test. From connector 1, the process determines if the connection is successful (140) and presents a dialog that the test will take up to 3 minutes to complete, for example (142). The process then reminds the user to prepare a sensor to be dipped into a liquid tray and to push a start button to initiate testing (144). The process animates the sensor being dipped into the liquid tray (146) and waits for liquid dispersion (148).

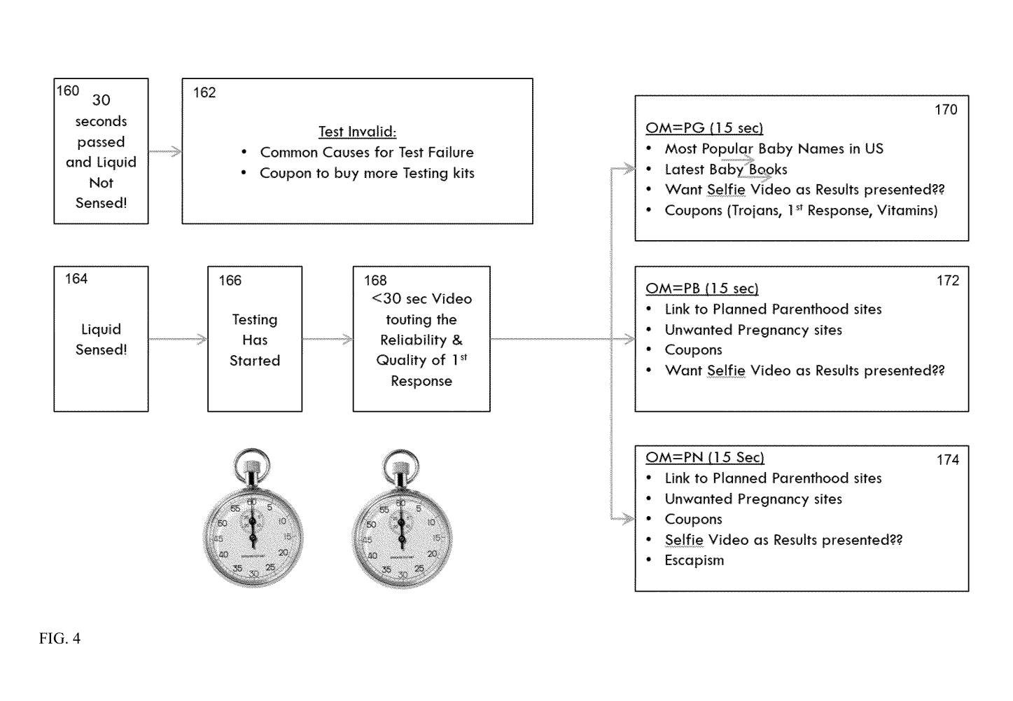

FIG. 4 shows an exemplary process to handle the results of the pregnancy test. In this figure, the test is invalidated if the sensor does not detect liquid within a predetermined period (such as 30 seconds) (160). The process displays common causes for the test failure and optionally provides coupons to buy more test kits (162). Alternatively, if liquid is sensed (164), the process determines the start of testing (166) and within a predetermined period, the process shows a video or other multimedia file on the reliability and quality of the test results (168). Next, depending on the OM setting, the process displays a number of suggestions to the user within a predetermined period (such as 15 seconds). For PG, the process shows the most popular baby names, latest baby books, selfie videos, and coupons for matching products) (170). In another case, for PB, the process links to Planned Parenthood sites, unwanted pregnancy sites, selfie videos, and suitable coupons, among others (172). For PN, the process links to Planned Parenthood sites, unwanted pregnancy sites, selfie videos, and shows escape/vacation options, for example (174).

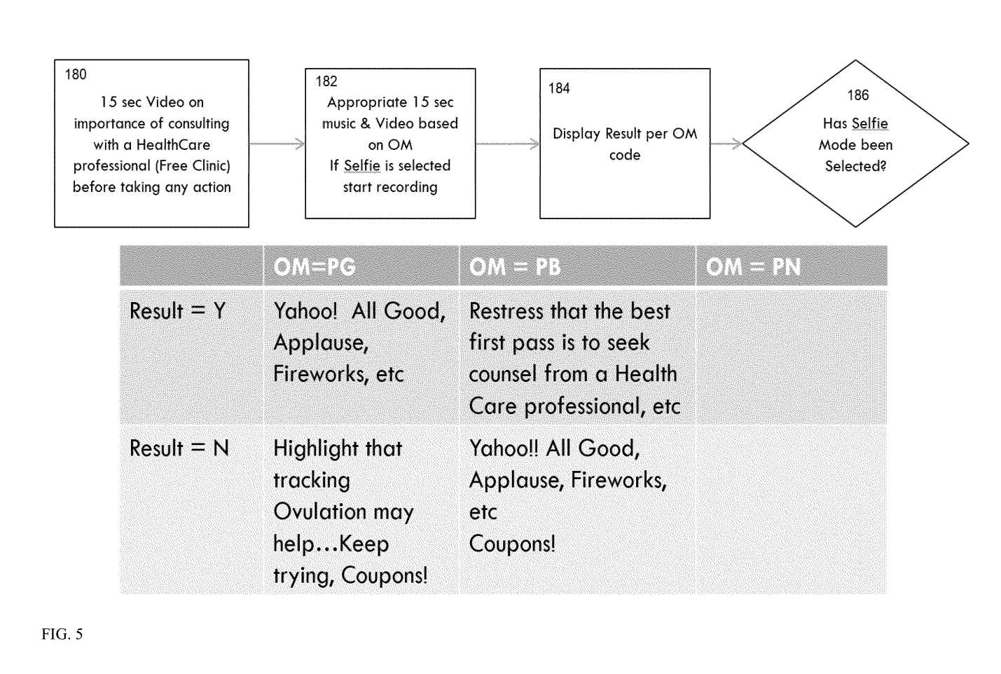

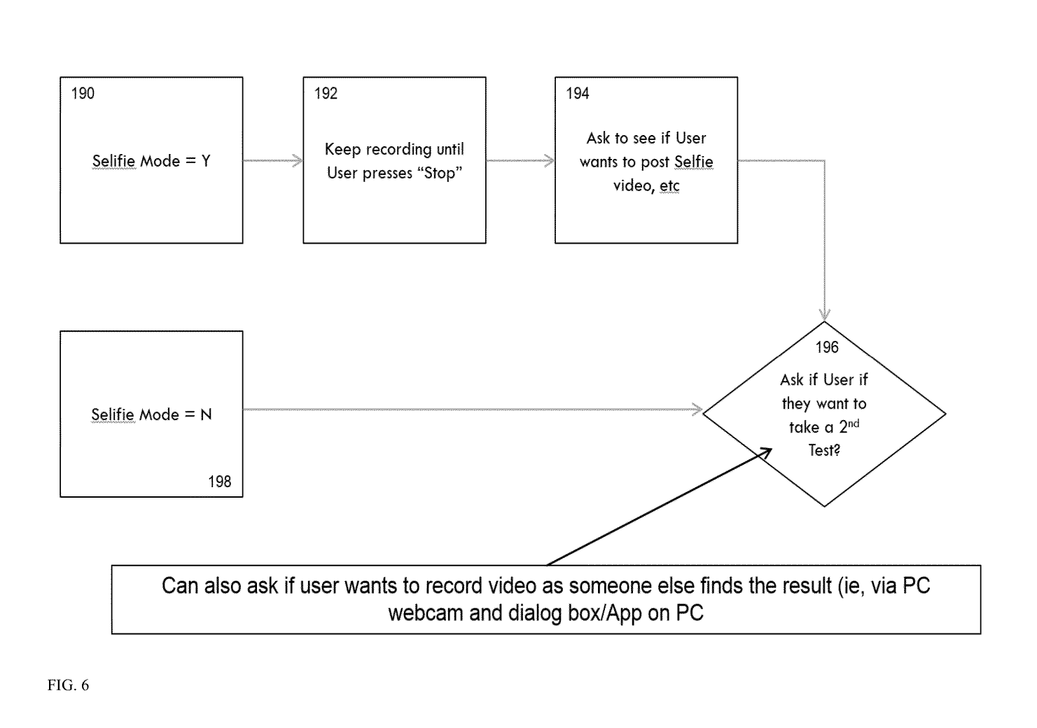

FIGS. 5 and 6 show an exemplary process to handle a positive pregnancy result. The process shows a video encouraging the user to consult with a doctor or suitable health-professional before taking any actions based on the test results (180). The process then plays music and video based on the OM setting (182), and if the selfie option is selected, the process starts recording (182). The result is displayed based on the OM code (184). For example, if OM is PG and the result is positive, the process displays cheers, applause, and firework, among others, and if the result is negative, the process highlights that tracking ovulation may help and provides coupons for suitable products. Correspondingly, if OM is PB and the result is positive, the process instructs the user to seek counsel from a doctor or health-professional, and if the result is negative, the process displays cheers, applause, and firework, and provides suitable coupons, among others.

The process then determines whether a selfie mode has been selected (186) and if so proceeds to FIG. 6, which shows an exemplary process to demonstrate a selfie use of the test device. Thus, if selfie mode is on (190), the process keeps recording until the user stops the recording (192) and then asks if the user wishes to post the video or image of the selfie (194). From this flow, or if the selfie mode is no (198), the process asks if the user wishes to take a second test (196). The process can also ask if the user wants to record the video as someone else who finds the result using a PC web cam, for example.

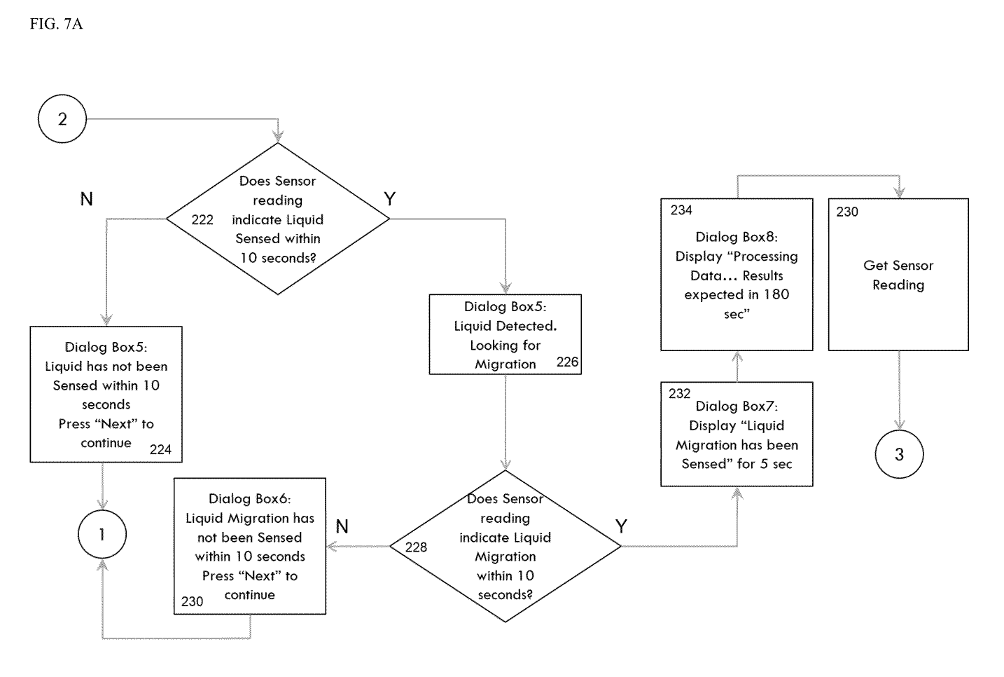

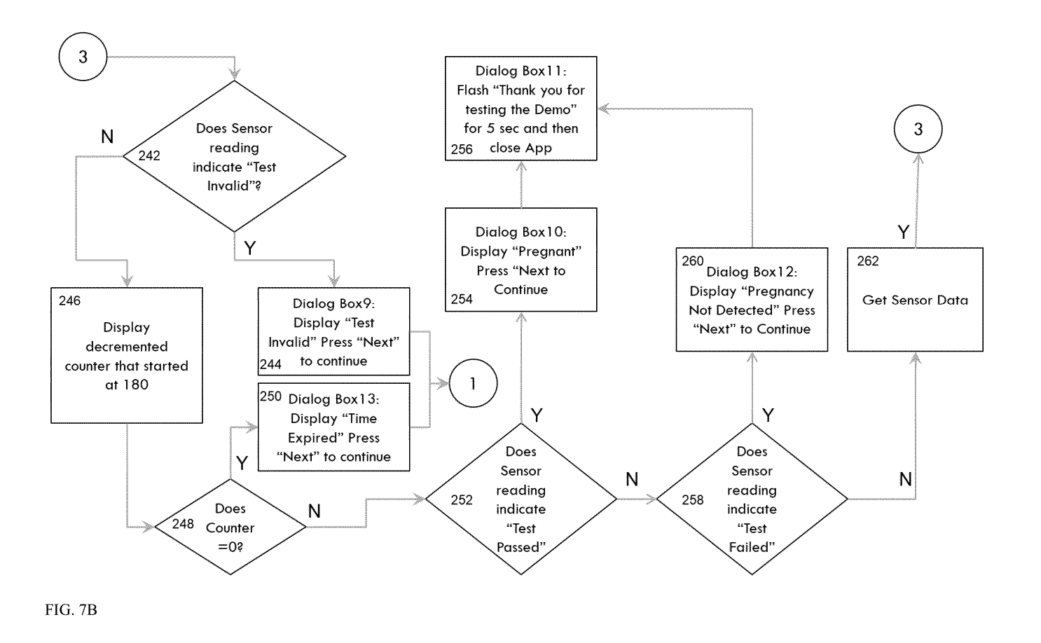

FIGS. 7A-7B shows an exemplary process operated by the test device. Turning now to the process of FIG. 7A, at the start (202), the user downloads the app (204) and launches the app on a smart phone (206). The process then requests the user to connect the smart phone with a test sensor device over a wireless connection (208). The process checks if the connection is successful (210) and if so the process indicates a successful connection and prompts the user to press a Start Test button or an Exit button on the user interface (212). If Exit is selected, the process jumps to connector 21. If the Start Test button is selected, the process prompts the user to push a Next button after liquid is applied to the sensor (214). If the user clicks on the Exit button, the process moves to 21 where it flashes a message “Thank you” (216) and returns to Welcome screen to prompt the user to press Connect or Close the app (208). From 210, if connection fails, the process displays a message on Connection Failure and prompts the user to replace battery and/or Bluetooth settings and thereafter continue on by pressing Next button (218).

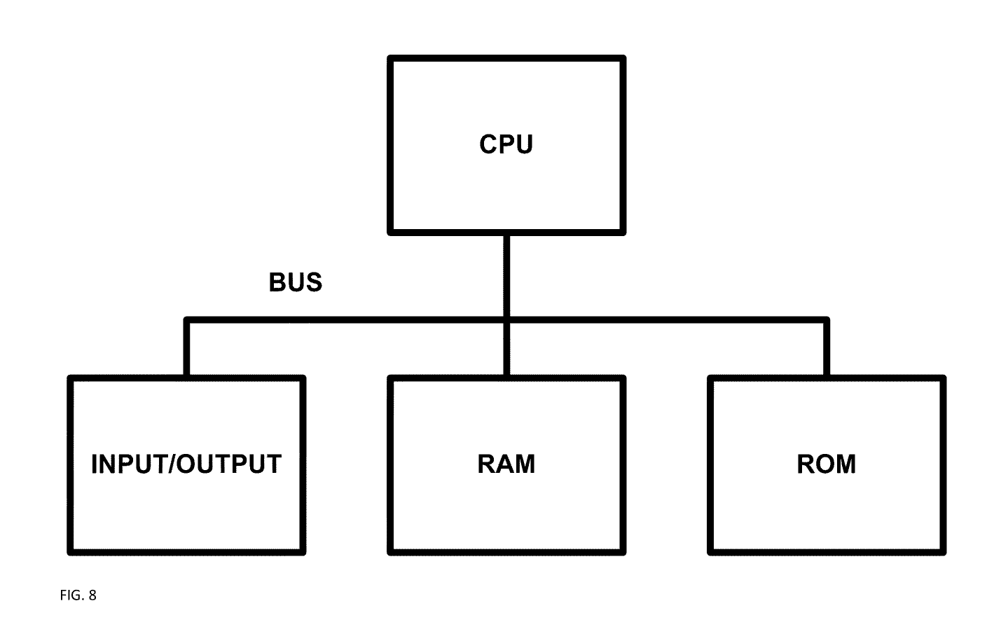

Referring now to FIG. 7B, from connector 22, the process checks if the sensor reading indicates that the liquid has been sensed within 10 seconds (222). If not, the process displays that liquid has not been detected and asks the user to press Next to continue (224) and upon that event jumps to connector 21 of FIG. 8A. Alternatively, if the liquid is detected in a timely manner, the process checks for migration (226). The process proceeds to check if the sensor reading indicates liquid migration within a predetermined period (such as 10 seconds) (228) and if not the process displays a message that liquid migration has not been sensed and prompts the user to click Next to continue (230). Otherwise, the process displays a message on the detection of the liquid migration (232), and notifies the user that results are imminent (234). The process then gets the sensor reading (236) and then jumps to connector 23 to FIG. 7C.

From connector 23, the process checks whether the sensor reading indicates an invalid test (242). If so, the process displays an invalid test message and prompts the user to click Next to continue (244) through connector 21. Otherwise, the process displays a decremented counter (246) and checks if the counter is zero (248) and if so the process displays that the time has expired (250) before receiving the Next button to jump through connector 21. From 248, if counter is positive, the process checks if the sensor indicates “test passed” (252) and if so the process displays a message indicating pregnancy (254) and flashes a message such as thank you before closing the app (256).

From 252, if the sensor detects a failed test, the process checks if the sensor reading indicates a test failure (258). And if so displays a dialog box indicating that pregnancy is not detected (260) and then proceeds to 256. Otherwise, the process gets sensor data (262) and jumps through connector 23 to 242.

FIG. 8 shows an exemplary test system. The system includes a processor, memory, I/O devices, and wireless transceiver(s) that can communicate pregnancy test data with transceiver(s) on a smart phone, for example. The device can include a camera to capture an image of the test strip and automatically determine change of color in the test strip.

The system may be implemented in hardware, firmware or software, or a combination of the three. Preferably the invention is implemented in multiple computer programs that execute on a programmable computer having a processor, a data storage system, volatile and non-volatile memory and/or storage elements, at least one input device and at least one output device.

Each computer program is tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

The invention has been described herein in considerable detail in order to comply with the patent Statutes and to provide those skilled in the art with the information needed to apply the novel principles and to construct and use such specialized components as are required. However, it is to be understood that the invention can be carried out by specifically different equipment and devices, and that various modifications, both as to the equipment details and operating procedures, can be accomplished without departing from the scope of the invention itself.

In view of the foregoing disclosure, some advantages of the present invention can be seen. For example, a novel pregnancy test device is disclosed.

While the preferred embodiment of the present invention has been described and illustrated, modifications may be made by one of ordinary skill in the art without departing from the scope and spirit of the invention as defined in the appended claims. For example, in a preferred embodiment of the present invention, the molded body is formed of high impact plastic; however any material known to one of ordinary skill in the art may be utilized to form the molded body. Additionally, the molded body in a preferred embodiment is constructed of a base and a cover fastened together; however the molded body could be formed differently as one of ordinary skill in the art might choose to form the molded body.

What is claimed is:

1. A method for detecting pregnancy or ovulation status, comprising:
establishing a wireless connection between an ovulation or pregnancy test sensor, a mobile device and cloud data servers;
displaying guidance information on the mobile device for a user to perform a pregnancy test;
displaying on the mobile device one or more multimedia presentations based on personalized options relating to pregnancy detection
capturing test data and retrieving historical data;
sharing test result with others and providing rich contextual information including education, medical references, test instruction, test diagnosis;
recording user reaction or reaction of others to the test results,
providing access to purchase consumables related to the test; and,
applying cloud computing to population data to improve test results.

2. The method of claim 1, detecting a wireless connection between a pregnancy test sensor and a mobile device and if the wireless connection is made if no connection is found, comprising displaying a help video or a settings menu to enable the wireless connection.

3. The method of claim 1, comprising determining if a selfie mode is selected and capturing and uploading a video of the user.

4. The method of claim 1, comprising detecting the presence of the liquid and check for liquid migration before getting a sensor reading.

5. The method of claim 4, comprising checking for an invalid test signal from the sensor reading.

6. The method of claim 5, comprising:
delaying for a predetermined period before checking for a test passed signal from the sensor reading;
indicating pregnancy if the test passed and if the test failed indicating no pregnancy and otherwise obtaining a new sensor reading.

7. The method of claim 1, wherein the mobile device comprises a smart phone and the wireless connection comprises a Bluetooth connection.

8. The method of claim 1, comprising capturing user reaction to key milestone events, recording the reactions and enabling the user to share the recording with others.

9. The method of claim 1, comprising enhancing sensor performance by using repeated measurements with potentially different thresholds that are set in a server on the cloud and enhancing accuracy of the pregnancy test by utilizing computational resources only available in the cloud server.

10. The method of claim 1, wherein the liquid is urine, comprising displaying guidance information on the mobile device for a user to dip a strip into a liquid and detecting at the sensor the presence of liquid on the strip and color change on the strip.

11. A system for detecting pregnancy, comprising:
a mobile device with a display and cellular connection and personal area network (PAN) wireless connection;
a test sensor coupled to the PAN; and
computer readable code for:
establishing a wireless connection between an ovulation or pregnancy test sensor, a mobile device and cloud data servers;
displaying guidance information on the mobile device for a user to perform a pregnancy test;
displaying on the mobile device one or more multimedia presentations based on personalized options relating to pregnancy detection capturing test data and retrieving historical data;
sharing test result with others and providing rich contextual information including education, medical references, test instruction, test diagnosis;
recording user reaction or reaction of others to the test results,
providing access to purchase consumables related to the test; and,
applying cloud computing to population data to improve test results.

12. The system of claim 11, comprising code for displaying a help video or a settings menu to enable the wireless connection if no connection is found.

13. The system of claim 11, comprising code for determining if a selfie mode is selected and capturing and uploading a video of the user.

14. The system of claim 11, comprising code for detecting the presence of the liquid and check for liquid migration before getting a sensor reading.

15. The system of claim 14, comprising code for checking for an invalid test signal from the sensor reading.

16. The system of claim 15, comprising code for delaying for a predetermined period before checking for a test passed signal from the sensor reading.

17. The system of claim 16, comprising code for indicating pregnancy if the test passed and if the test failed indicating no pregnancy and otherwise obtaining a new sensor reading.

18. The system of claim 11, wherein the wireless connection comprises a Bluetooth connection.

19. The system of claim 11, wherein the mobile device comprises a smart phone.

20. The system of claim 11, wherein the liquid is urine, comprising code for checking for concentration of hCG in urine by holding the strip vertically, dipping the strip into the urine and waiting for colored bands to appear.
